(19) United States
(12) Patent Application Publication
(10) Pub. No.: US 2011/0092604 A1
(43) Pub. Date: Apr. 21, 2011

(54) FRAGRANCE SUBSTANCE MIXTURES COMPRISING ISOLONGIFOLANOL AND MUSK

(75) Inventors: Ingo Wöhrle, Bremen (DE); Marcus Eh, Holzminden (DE); Marinus Cornelius Van Dijk, Dusseldorf (DE)

(73) Assignee: SYMRISE AG, Holzminden (DE)

(21) Appl. No.: 12/907,725

(22) Filed: Oct. 19, 2010

(30) Foreign Application Priority Data
Oct. 19, 2009 (EP) 09 173 455.8

(51) Int. Cl. C11D 3/50; A61K 8/34; A61K 8/35; A61Q 13/00

(52) U.S. Cl. 514/772; 512/19; 512/15; 510/104

(57) ABSTRACT

The present invention relates to fragrance substance mixtures, in particular perfume oils, comprising
(a) compound (A)

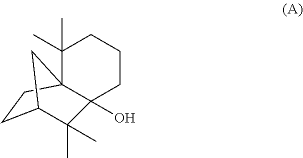

and
(b) at least one musk fragrance substance
wherein
the mass ratio of the total quantity of the musk fragrance substance or substances of component (b) to compound (A) is greater than or equal to 1:1.

The present invention also relates to perfumed products comprising fragrance substance mixtures of this type according to the invention, in particular perfume oils, and to a process for the production of the fragrance substance mixtures and perfume oils according to the invention.

The present invention relates to fragrance substance mixtures, in particular perfume oils, comprising

(a) compound (A) (isolongifolanol)

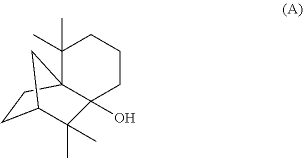

and
(b) at least one musk fragrance substance
where
the mass ratio of the total quantity of the musk fragrance substance or substances of component (b) to compound (A) (component (a)) is greater than or equal to 1:1.

The present invention also relates to perfumed products comprising fragrance substance mixtures of this type according to the invention, in particular perfume oils, and to a process for the production of the fragrance substance mixtures and perfume oils according to the invention.

EP 1 215 189 describes a process for the production of compound (A) (isolongifolanol) and describes its olfactory characteristics. In that document, compound (A) is characterized as a fragrance substance with woody and patchouli notes which also has camphoric, ambered and green aspects. Furthermore, it is stated in EP 1 215 189 that compound (A) is suitable to be incorporated into woody and spicy compositions and to adjust natural patchouli oils. A general list of numerous fragrance substances also mentions, inter alia, musk fragrance substances, without any detailed information being provided on specific proportions or specific olfactory effects in combinations therewith. The only perfume oil specifically mentioned there comprises isolongifolenol (not compound (A)) and the macrocyclic musk fragrance substance (Globalid®) in a ratio of 400:2.

Compound (A) is known from JP 58-022450 and JP 60-010007 B4. In the process described therein, 5% of the compound is obtained by hydrogenating isolongifolene oxide in a multi-component mixture.

The reaction of isolongifolene oxide with lithium aluminum hydride is described in J. Agric. Food. Chem. 1994, 42, 138-142. Small amounts (<100 mg) of compound (A) were obtained in this manner.

In Indian Perfumer 2005, 49(4), 453-455, compound (A) is described as a component of the essential oil of Cyperus rotundus L. tubers. This essential oil does not comprise any musk fragrance substances.

Perfume oils and perfumed products comprising one or more musk fragrance substances are known to a person skilled in the field of perfumery, see, for example EP 1 552 814.

Musk fragrance substances are known to a person skilled in the art and they play a very important part in perfumery. In perfumery, there is always a need to emphasize (bring out) specific olfactory aspects of a fragrance substance or of a fragrance substance mixture and, in the case of musk fragrance substances, this applies in particular to the erogenous, masculine and animalic odor character thereof.

This object is achieved by a fragrance substance mixture according to the invention, preferably by a perfume oil, comprising

(a) compound (A)

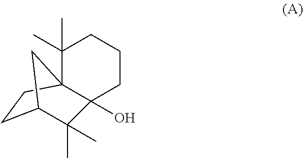

and
(b) at least one musk fragrance substance,
wherein
the mass ratio of the total quantity of the musk fragrance substances of component (b) to compound (A) (component (a)) is greater than or equal to 1:1, preferably from 1:1 to 50:1.

### Component (a) of a Fragrance Substance Mixture According to the Invention

Compound (A) (isolongifolanol; IUPAC name: 2,2,7,7-Tetramethyltricyclo[6.2.1.0^{1.6}]undecan-6-ol) of the structural formula given above is known from the literature and can be produced, for example, as described in EP 1 215 189 (see above).

### Component (b) of a Fragrance Substance Mixture According to the Invention

The musk fragrance substance or substances of component (b) are fragrance substances which have a musk odor. Fragrance substances of this type are known to a person skilled in the art, since musk is a very important odor direction in perfumery.

According to the invention, the mass ratio of the total quantity of musk fragrance substances, i.e. of component (b), to compound (A) (component (a)), in other words the total weight ratio of (b) to (a) is greater than or equal to 1:1, preferably from 1:1 to 50:1, more preferably from 25:1 to 1:1, particularly preferably from 4:3 to 20:1 and very particularly preferably from 4:3 to 15:1. The total weight ratio of component (b) to component (a) is most preferably from 2:1 to 12:1, in particular from 2:1 to 10:1, and in many further preferred embodiments the total weight ratio is from 5:2 to 8:1.

Mixtures according to the invention comprising compound (A) and one or more musk fragrance substances, in particular those from the following Table 1, in which the mass ratio of the total quantity of musk fragrance substances (b) to compound (A) is greater than or equal to 1, emphasize (bring out) the erogenous, masculine and animalic odor character which is peculiar to all musk compounds.

The musk fragrance substance or substances (b) are preferably selected from the group of macrocyclic musk fragrance substances, nitro-musk fragrance substances (fragrance musk fragrance substances with nitro group(s)), polycyclic musk fragrance substances and/or alicyclic musk fragrance substances.

Fragrance substance mixtures which are preferred according to the invention, preferably perfume oils, comprise two, three or more different musk fragrance substances as component (b), each of the musk fragrance substances preferably being selected from the group of macrocyclic musk fragrance substances, nitro-musk fragrance substances (fragrance musk fragrance substances with nitro group(s)), polycyclic musk fragrance substances and/or alicyclic musk fragrance substances, independently of the other musk fragrance substances.

In the context of the present invention, the musk fragrance substance(s) are preferably selected from the following Table 1:

TABLE 1

| TYPE | Product/Brand name | Name/CAS Name |
|---|---|---|
| MACRO | EXALTENONE | 4-Cyclopentadecen-1-one (4Z)-; 4- |
| | | Cyclopentadecen-1-one |
| MACRO | CIVETONE | 9-Cycloheptadecen-1-one, (9Z)- |
| MACRO | CYCLOHEXADECANOLIDE, | Oxacycloheptadecan-2-one, |
| | DIHYDROAMBRETTOLIDE | ω-Hexadecanolide |
| MACRO | ETHYLENE DODECANDIOATE | 1,4-Dioxacyclohexadecane-5,16-dione |
| MACRO | GLOBALIDE ® | Oxacyclohexadecen-2-one; 15- |
| | | Pentadec-(11/12)-enolide |
| MACRO | ETHYLENE BRASSYLATE | 1,4-Dioxacycloheptadecane-5,17- |
| | | dione |
| MACRO | MUSCONE | 3-Methy-cyclopentadecanone |
| MACRO | AMBRETTOLIDE | Oxacycloheptadec-10-en-2-one |
| MACRO | MUSCENONE | 3-Methyl-cyclopentadecenone |
| MACRO | VELVIONE ®, AMBRETONE | 5-Cyclohexadecen-1-one |
| MACRO | AURELIONE ® | 7/8-Cyclohexadecen-1-one |
| MACRO | GLOBANONE ® | 8-Cyclohexadecen-1-one |
| MACRO | ISOMUSCONE ® | Cyclohexadecanone |
| MACRO | EXALTOLIDE, MACROLIDE ® | Oxacyclohexadecan-2-one |
| MACRO | COSMONE ® | 3-Methyl-(5E/Z)-cyclotetradecen-1-one |
| POLY | TRASEOLIDE ® | 1-[2,3-Dihydro-1,1,2,6-tetramethyl-3- |
| | | (1-methylethyl)-1H-inden-5-yl]- |
| | | ethanone |
| POLY | PHANTOLIDE ® | 1-(2,3-Dihydro-1,1,2,3,3,6-hexamethyl- |
| | | 1H-inden-5-yl)-ethanone |
| POLY | TONALIDE ® | 1-(5,6,7,8-Tetrahydro-3,5,5,6,8,8- |
| | | hexamethyl-2-naphthalenyl)-ethanone |
| POLY | CRYSOLIDE | 1-[6-(1,1-Dimethylethyl)-2,3-dihydro- |
| | | 1,1-dimethyl-1H-inden-4-yl]-ethanone |
| POLY | CHROMANOLIDE ® | Tetradecanoic acid, 1-methylethyl |
| | | ester; Cyclopenta[g]-2-benzopyrane, |
| | | 1,3,4,6,7,8-hexahydro-4,6,6,7,8,8- |
| | | hexamethyl- |
| POLY | GALAXOLIDE ® | Cyclopenta[g]-2-benzopyrane, |
| | | 1,3,4,6,7,8-hexahydro-4,6,6,7,8,8- |
| | | hexamethyl- |
| ALICYC | HELVETOLIDE ® | 1-Propanol, 2-[1-(3,3- |
| | | dimethylcyclohexyl)ethoxy]-2-methyl-, |
| | | 1-propanoate |
| NITRO | MOSKENE | 2,3-Dihydro-1,1,3,3,5-pentamethyl-4,6- |
| | | dinitro-1H-indene |
| NITRO | MUSK TIBETENE | 1-(1,1-Dimethylethyl)-3,4,5-trimethyl- |
| | | 2,6-dinitrobenzene |
| NITRO | ORINOX | 1-[4-(1,1-Dimethylethyl)-2,6- |
| | | dimethylphenyl]-ethanone |
| NITRO | MUSK XYLENE | 1-(1,1-Dimethylethyl)-3,5-dimethyl- |
| | | 2,4,6-trinitrobenzene |
| NITRO | MUSK KETONE | 1-[4-(1,1-Dimethylethyl)-2,6-dimethyl- |
| | | 3,5-dinitrophenyl]-ethanone |
| NITRO | MUSK ALPHA | 1,3-Dibromo-2-methoxy-4-methyl-5- |
| | | nitrobenzene |

MACRO = macrocyclic musk fragrance substances
NITRO = nitro-musk fragrance substances
POLY = polycyclic musk fragrance substances
ALICYC = alicyclic musk fragrance substances

Further preferred as part of component (b) are polycyclic and/or macrocyclic musk fragrance substances, macrocyclic musk fragrance substances in particular having proved to be particularly advantageous in the context of the invention which, in turn, are preferably selected from the group consisting of macrocyclic C₁₄-C₁₈ ketones and macrocyclic C₁₄-C₁₈ lactones, the ketone or lactone having a ring size of from 15 to 17 ring atoms and having no oxygen atoms or one or two oxygen atoms in the ring.

Most preferred are fragrance substance mixtures according to the invention, the component (b) of which comprises or is selected from: 3-methylcyclopentadecenone (Muscenone), 15-pentadec-(11/12)-enolide (Globalide)®, ethylene brassylate, oxacyclohexadecan-2-one (Macrolide®), cyclohexadecanone (Isomuscone®), 8-cyclohexadecanone (Globanone®), (7/8)-cyclohexadecanone (Aurelione®) and mixtures thereof.

In a preferred embodiment, component (b) is selected from the group consisting of 15-pentadec-(11/12)-enolide (Globalide®), ethylene brassylate and oxacyclohexadecan-2-one (Macrolide®) and mixtures thereof.

A process according to the invention for emphasizing (bringing out) the erogenous, masculine and/or animalic odor impression of one or more musk compounds comprises the following step:
mixing one or more musk compounds, preferably selected from the compounds mentioned above as being preferred, of component (b), with a quantity of compound (A) which is sufficient to emphasize the erogenous, masculine and/or animalic odor impression of one, more or all the musk compounds of component (b).

The animalic character of the musk fragrance substances is harmonized and rounded off in a particular manner by the addition of compound (A) with the preferably one, two, three, four, five, six, seven, eight, nine, ten or more further fragrance substances which are preferably a component of a fragrance substance mixture according to the invention, preferably of a perfume oil which is preferred according to the invention.

This produces a harmonious, elegant and valuable, that is, harmonious and complex odor impression.

This applies in particular to the further fragrance substances, preferably present, of the base (the base notes) of a fragrance substance mixture preferred according to the invention, preferably of a perfume oil preferred according to the invention, because there the harmonization and rounding-off produces a harmonious, elegant and creamy and thus very valuable odor impression. Base notes which are preferred in this connection are the fragrance substances described below as component (c).

Furthermore, this effect applies in particular to the further fruity fragrance substances, preferably present, of a fragrance substance mixture preferred according to the invention, preferably of a perfume oil preferred according to the invention, because there the harmonization and rounding-off produces a harmonious and complex and thus very valuable odor impression. Fruity fragrance substances which are preferred in this connection are the fragrance substances described below as component (d).

### Optional Component (c) of a Fragrance Substance Mixture According to the Invention:

In addition to components (a) and (b), a fragrance substance mixture according to the invention, preferably a perfume oil according to the invention, preferably comprises as a further component
(c) one, two, three, four, five or more fragrance substances with a molecular weight of greater than or equal to 190 g/mol which are not compound (A) (component (a)) and are not part of component (b).

Examples of fragrance substances having a molecular weight ≧190 g/mol, which can be part of the optional component (c) are known to a person skilled in the art and can be found, for example, in S. Arctander, Perfume and Flavor Materials, Vol. I and II, Montclair, N.J., 1969, private publishing house or H. Surburg, J. Panten, “Common Fragrance and Flavor Materials”, 5^{th}. Ed., Wiley-VCH, Weinheim 2006.

The fragrance substance or substances of component (c) of a fragrance substance mixture according to the invention have a molecular weight of greater than or equal to 190 g/mol, preferably a molecular weight of greater than or equal to 195 g/mol.

The fragrance substances of component (c) are to be considered as base notes of a fragrance substance mixture according to the invention or of a perfume oil according to the invention.

Fragrance substance mixtures according to the invention, in particular perfume oils according to the invention comprising compound (A), one or more musk fragrance substances (component (b)) and component (c) of the base notes exhibit the harmonization and rounding off, already described above, thereby producing an elegant, creamy and thus very valuable odor impression. This applies in particular to the base notes which are defined in the following as being preferred and in particular to the explicitly mentioned base notes.

The molecular weight of the fragrance substances of component (c) is preferably from 200 to 300 g/mol, more preferably from 200 to 280/mol, particularly preferably from 200 to 265 g/mol and most preferably from 200 to 245 g/mol, since with fragrance substances of this type, combined with component (a), in particular with the fragrance substances of components (a) and (b), the particular olfactory effects of the present invention are achieved most strongly and clearly.

Fragrance substance mixtures preferred according to the invention, preferably perfume oils according to the invention are those in which component (c) comprises one, two, three or more fragrance substances from the group consisting of the following: (the molecular weights (MW) and, if appropriate, conventional brand or product names are stated in brackets):

alpha-n-amylcinnamaldehyde (MW=202.30), alpha-iso-amylcinnamaldehyde (MW=202.30), alpha-n-hexylcinnamaldehyde (MW=216.32), alpha-iso-hexylcinnamaldehyde (MW=216.32), benzyl salicylate (MW=228.25), cis-3-hexenylsalicylate (MW=220.27), isoamyl salicylate (MW=208.26), hexyl salicylate (MW=222.28), 2-methyl-3-(4-tert-butylphenyl)propanal (MW=204.31; Lilial®), 2,3,8,8-tetramethyl-1,2,3,4,5,6,7,8-octahydro-2-naphthalenylmethylketone (MW=234.38, Iso E Super®), 1-[1,2,3,4,6,7,8,8a-octahydro-1,2,8,8-tetramethylnaphthalen-2-yl]ethanone (MW=234.38, Iso E Super®), methyl dihydrojasmonate (MW=226.32, Hedione®), linalyl acetate (MW=196.29), ethyl linalyl acetate (MW=210.31), Nerolidol (MW=222.37), Farnesol (MW=222.37), cedrylmethylether (MW=236.40, Cedramber), cedrylmethylketone (MW=246.39), cedrylacetate (MW=264.41), (4aR,5R,7aS,9R)-octahydro-2,2,5,8,8,9a-hexamethyl-4H-4-a,9-methanoazuleno(5,6-d)-1,3-dioxole) (MW=278.44, Ambrocenide®), 1′,1′,5′,5′-tetramethylhexahydro-spiro[1,3-dioxolane,2,8′(5′H)-[2H-2.4a]-methanonaphthalene (MW=264.41, Ysamber® K), 2-methyl-4-(2,2,3-trimethyl-3-cyclopenten-1-yl)butanol (MW=196.34, brahmanol), 5-(2,2,3-trimethyl-3-cyclopenten-1-yl)-3-methylpentan-2-ol (MW=210.36, Sandalore®), 2-ethyl-4-(2,2,3-trimethyl-3-cyclopenten-1-yl)-2-buten-1-ol (MW=208.35, Sandranol®), 3-methyl-5-(2,2,3-trimethyl-3-cyclopenten-1-yl)-4-penten-2-ol (MW=208.35, Ebanol®), 3,3-dimethyl-5-(2,2,3-trimethyl-3-cyclopenten-1-yl)-4-penten-2-ol (MW=222.37, Polysantol®), 3-isocamphylcyclohexanol (MW=236.40, Sandel 80®), 1-(2,2,6-trimethylcyclohexyl)hexan-3-ol (MW=226.41, Timberol®), cyclododecylmethylether (MW=198.35, Palisandin), (ethoxymethoxy)cyclododecane (MW=242.41, Boisambrene Forte®), 1-methyl-4-(4-methyl-3-penten-1-yl)-3-cyclohexencarboxaldehyde (MW=206.33, Precyclemone B®), 4-(4-hydroxy-4-methylpentyl)-3-cyclohexenecarboxaldehyde (MW=210.32, Lyral®), 2-methyl-4-(2,2,6-trimethyl-1-cyclohexen-1-yl)-2-butenal (MW=206.33, Boronal), decahydro-beta-naphthylacetate (MW=196.29), 4,7-methano-3a,4,5,6,7,7a-hexahydro-5(-6)-indenylacetate (MW=192.26, Herbaflorat®), allyl-3-cyclohexylpropionate (MW=196.29), allylcyclohexyloxyacetate (MW=198.26, Isoananat®), Citraldiethylacetal (MW=226.36), benzylbenzoate (MW=212.25), benzylcinnamate (MW=238.29), 3a,6,6,9a-tetramethyldodecahydronaphtho[2,1-b]furan (MW=236.40, Ambroxid®), alpha-Iron (MW=206.33), beta-Iron (MW=206.33), alpha-n-methylionone (MW=206.33), beta-n-methylionone (MW=206.33), alpha-isomethylionone (MW=206.33), beta-isomethylionone (MW=206.33) and allylionone (MW 232.35).

Particularly preferred fragrance substance mixtures according to the invention, preferably perfume oils according to the invention, are those in which component (c) is or comprises
(c) (i) 2,3,8,8-tetramethyl-1,2,3,4,5,6,7,8-octahydro-2-naphthalenylmethylketone and/or 1-[1,2,3,4,6,7,8,8a-octahydro-1,2,8,8-tetramethylnaphtalen-2-yl]ethanone
and/or
(c) (ii) methyl dihydrojasmonate.

Mixtures comprising both compounds of component (c) (i) are known, for example, under the name of Iso E Super® and are commercially available.

Fragrance substance mixtures according to the invention, in particular perfume oils according to the invention, comprising compound (A), one or more musk fragrance substances (component (b)) and (c) (i) 2,3,8,8-tetramethyl-1,2,3,4,5,6,7,8-octahydro-2-naphthalenylmethylketone and/or 1-[1,2,3,4,6,7,8,8a-octahydro-1,2,8,8-tetramethylnaphtalen-2-yl]ethanone, in particular in the mass ratios stated in the following, produce, in addition to the harmonization and rounding off effects, a classic vetiver effect which makes the entire composition appear more valuable. The term “vetiver effect” is understood by a person skilled in the art (perfumer) as follows: the odor is reminiscent of vetiver oil which is characterized by a sumptuous dry woody odor with grassy and rooty aspects.

Component (c) (ii) can be present as a cis- or trans-isomer or as a mixture of these isomers. In the context of the present invention, a content of cis-methyl dihydrojasmonate of more than 30% by weight is preferred, more preferred is a content of more than 60% by weight, particularly preferred is a content of more than 75% by weight and in some cases even more than 90% by weight, in each case based on the total quantity of methyl dihydrojasmonate. Methyl dihydrojasmonate is commercially available in different isomer mixtures, for example under the name of Hedione®, Hedione® HC).

Fragrance substance mixtures according to the invention, in particular perfume oils according to the invention, comprising compound (A), one or more musk fragrance substances (component (b)) and (c) (ii) methyl dihydrojasmonate, in particular in the mass ratios stated in the following, produce, in addition to the harmonization and rounding off effects, a “Fleur d'Orange” effect and emphasize in a unique manner the transparency of methyl dihydrojasmonate which makes the entire composition appear more valuable.

The term “Fleur d'Orange” effect is understood by a person skilled in the art (perfumer) as follows: the odor is reminiscent of natural “Fleur d'Orange” [“orange flower”] which is characterized by a beguiling floral odor of white blossom and aspects of fresh oranges.

The term “transparency” of a fragrance composition is understood by a person skilled in the art (perfumer) to be that the odor of a fragrance substance mixture or of a perfume oil has a watery and ozonic clarity.

A further preferred embodiment relates to a fragrance substance mixture according to the invention, preferably a perfume oil according to the invention, in which the mass ratio of the total quantity of component (c) (i) 2,3,8,8-tetramethyl-1,2,3,4,5,6,7,8-octahydro-2-naphthalenylmethylketone and 1-[1,2,3,4,6,7,8,8a-octahydro-1,2,8,8-tetramethylnaphtalen-2-yl]ethanone to compound (A) (component (a)) is greater than or equal to 1:1, preferably from 4:3 to 10:1, particularly preferably from 2:1 to 8:1.

A further preferred embodiment relates to a fragrance substance mixture according to the invention, preferably a perfume oil according to the invention, in which the mass ratio of the total quantity of component (c) (ii) methyl dihydrojasmonate to compound (A) (component (a)) is greater than or equal to 1:1, preferably from 2:1 to 15:1, particularly preferably from 3:1 to 10:1.

### Optional Component (d) of a Fragrance Substance Mixture According to the Invention:

A fragrance substance mixture according to the invention, preferably a perfume oil according to the invention, comprises, in addition to components (a) and (b) and optionally (c) as a further component (d) one, two, three or more fruity fragrance substances, preferably selected from the group of fragrance substances stated in the following Table 2, wherein the fruity fragrance substance or substances are not part of components (a) and (b) and are not a component of optional component (c).

A fragrance substance mixture according to the invention, preferably a perfume oil according to the invention, preferably comprises a sensorially (olfactory) effective quantity of component (d), preferably in a quantity which is sufficient to impart a fruity note to a fragrance substance mixture according to the invention, preferably to a perfume oil according to the invention.

Fragrance substance mixtures according to the invention, in particular perfume oils according to the invention, comprising compound (A), one or more musk fragrance substances (component (b)) and one or more fruity fragrance substances (component (d)) exhibit the olfactory effects which have already been described above, thereby producing a harmonious and complex and thus very valuable odor impression. This applies in particular to the fragrance substances defined in the following as being preferred and to the explicitly mentioned fruity fragrance substances.

In a preferred embodiment, a fragrance substance mixture according to the invention, preferably a perfume oil according to the invention comprises, in addition to components (a) and (b), component (c) and also component (d).

If a fragrance substance mixture according to the invention, a perfume oil according to the invention or a perfumed product according to the invention comprises a component (d) which simultaneously also fulfills the criteria of component (c), this component (d) is considered, particularly for quantitative considerations, as component (c) and is associated therewith.

Examples of fruity fragrance substances which can be part of the optional component (d) are known to a person skilled in the art and can be found, for example, in S. Arctander, Perfume and Flavor Materials, Vol. I and II, Montclair, N.J., 1969, private publishing house or H. Surburg, J. Panten, “Common Fragrance and Flavor Materials”, 5^{th}. Ed., Wiley-VCH, Weinheim 2006.

The fruity fragrance substance or substances of component (d) are preferably selected from the group of fragrance substances listed in the following Table 2.

TABLE 2
Preferred fruity fragrance substances of component (d)

| Substance | Fruit note | Structure |
|---|---|---|
| 2-Methyl-butyric acid ethyl ester (ethyl-2- methylbutyrate) | Apple | 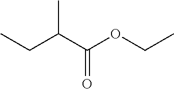 |
| 4-(p-Hydroxyphenyl)-2- butanone | Raspberry | 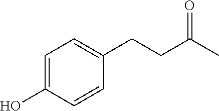 |
| Ethyl-3-methyl-3- phenylglycidate | Strawberry | 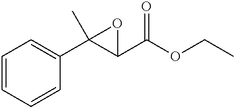 |
| Butyric acid isoamyl ester (Isoamyl butyrate) | Banana | 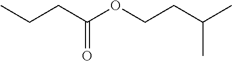 |
| Acetic acid isoamyl ester (Isoamyl acetate) | Banana | 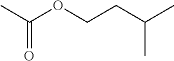 |
| Acetic acid-n-butyl ester (Butyl acetate) | Apple | 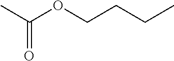 |
| Butyric acid ethyl ester (Ethyl butyrate) | Pineapple | 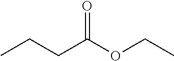 |
| 3-Methyl-butyric acid ethyl ester (Ethyl isovalerate) | Blueberries | 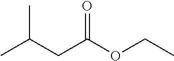 |
| n-Hexanoic acid ethyl ester (Ethyl caproate) | Pineapple | 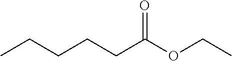 |
| n-Hexanoic acid allyl ester (2-propenylhexanoate) | Pineapple | 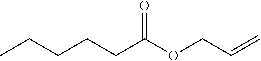 |
| Ethyl-2-trans-4-cis- decadienoate | Pear | 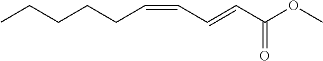 |
| 1,1-Dimethoxy-2,2,5- trimethyl-4-hexane | Grapefruit | 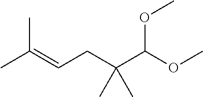 |
| 2,6-Dimethyl-5-hepten-1- al | Melon | 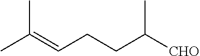 |
| Gamma-Undecalactone | Peach | 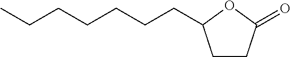 |
| Gamma-Nonalactone | Coconut | 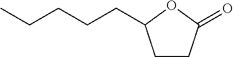 |
| D-limonene | Citrus (e.g. Orange, Lemon) | 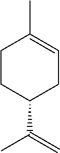 |
| Vanillin | Vanilla, sweet | 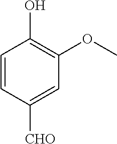 |
| Ethyl vanillin | Vanilla, sweet | 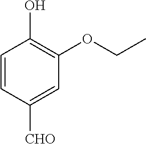 |
| Maltol | Caramel, sweet | 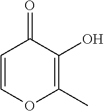 |
| Ethyl maltol | Caramel, very sweet | 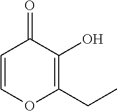 |

The fruity fragrance substances of component (d) can be of a natural or synthetic origin. If of a natural origin, they can be part of a corresponding essential oil or can be concentrated or isolated from such a corresponding essential oil. Thus, for example D-limonene is used in an embodiment preferred according to the invention in the form of orange peel oil, lemon oil, lime oil, bergamot oil, mandarin oil or grapefruit oil.

In a preferred embodiment of the present invention, red-fruited fragrance substances are used as component (d), the preferred red-fruited fragrance substances preferably having the odor of strawberries, raspberries, blackberries, redcurrants, bilberries (cassis), elderberries, cherries, red grapes and/or plums.

A fragrance substance mixture according to the invention, a perfume oil according to the invention or a product according to the invention is preferred, preferably in one of the embodiments stated above as being preferred, for which:
the mass ratio of the total quantity of musk fragrance substances of component (b) to component (a) is greater than or equal to 1:1, preferably from 1:1 to 50:1, more preferably from 25:1 to 1:1, particularly preferably from 4:3 to 20:1, very particularly preferably from 4:3 to 15:1, most preferably from 2:1 to 12:1, in particular from 2:1 to 10:1, said ratio being from 5:2 to 8:1 in further preferred embodiments,
and/or
the mass ratio of the total amount of component (c) (i) 2,3,8,8-tetramethyl-1,2,3,4,5,6,7,8-octahydro-2-naphthalenylmethylketone and 1-[1,2,3,4,6,7,8,8a-octahydro-1,2,8,8-tetramethylnaphtalen-2-yl]ethanone to compound (A) (component (a)) is greater than or equal to 1:1 and is preferably from 4:3 to 10:1, particularly preferably from 2:1 to 8:1,
and/or
the mass ratio of the total quantity of component (c) (ii) methyl dihydrojasmonate to compound (A) (component (a)) is greater than or equal to 1:1 and is preferably from 2:1 to 15:1, particularly preferably from 3:1 to 10:1,
and/or
a sensorially (olfactorily) effective quantity of component (d), preferably selected from the fruity fragrance substances mentioned in Table 2, is comprised, preferably in a quantity which is sufficient to impart a fruity note to the fragrance substance mixture, to the perfume oil or to the product,
   in each case based on the entire fragrance substance mixture or the entire perfume oil or product.

The preferred embodiments and/or mass ratios mentioned in respect of components (a), (b), (c) and (d) are preferably adjusted simultaneously.

In view of what has been stated above, it can thus be maintained that compound (A), together with the musk fragrance substance or substances of component (b) advantageously emphasizes (brings out) specific olfactory aspects of these fragrance substances when combined with other fragrance substances.

A process according to the invention for emphasizing an odor with a woody, vetiver, creamy, earthy, fresh, spicy, mossy, (red) fruity, transparent and/or Fleur d'Orange note comprises the following step:
mixing
   (i) one or more fragrance substances, preferably selected from the compounds, mentioned above as being preferred, of components (c) and/or (d), having one, more or all the woody, vetiver, creamy, earthy, fresh, spicy, mossy, (red) fruity, transparent and/or Fleur d'Orange notes,
   and
   (ii) one or more musk fragrance substances of component (b), preferably selected from the musk fragrance substances, described above as being preferred, of component (b),
with a quantity of compound (A) (component (a)) which is sufficient to emphasize the odor impression of the fragrance substance or substances which produce one, more or all the woody, vetiver, creamy, earthy, fresh, spicy, mossy, (red) fruity, transparent and/or Fleur d'Orange notes,
   or
   (ii) a fragrance substance mixture according to the invention, preferably a perfume oil according to the invention, preferably in one of the embodiments described above as being preferred, in a quantity which is sufficient to emphasize the odor impression of the fragrance substance or substances which produce one, more or all the woody, vetiver, creamy, earthy, fresh, spicy, mossy, (red) fruity, transparent and/or Fleur d'Orange notes.

The information provided above in respect of preferred fragrance substance mixtures, perfume oils and products preferred according to the invention applies accordingly to the processes according to the invention.

Fragrance substance compositions (fragrance substance mixtures) according to the invention, preferably perfume oils according to the invention, can be used in liquid form, undiluted or diluted with a solvent for perfuming or aromatization. Suitable solvents include in particular ethanol, glycerin, 1,2-propylene glycol, 1,2-butylene glycol, dipropylene glycol, diethyl phthalate, triethyl citrate, isopropyl myristate and triacetin.

Substances with which fragrance substance mixtures according to the invention, in particular perfume oils can be combined are:

preservatives, preferably those mentioned in US 2006/0089413, abrasives, anti-acne agents and sebum-reducing agents, preferably those mentioned in WO 2008/046791, skin anti-ageing agents, preferably those mentioned in WO 2005/123101, anti-bacterial agents, anti-cellulite agents, anti-dandruff agents, preferably those mentioned in WO 2008/046795, anti-inflammatory agents, irritation-prevention agents, anti-irritants (anti-inflammatory, irritation-blocking and irritation-preventing agents), preferably those mentioned in WO 2007/042472 and US 2006/0089413, antimicrobial agents, preferably those mentioned in WO 2005/123101, antioxidants, preferably those mentioned in WO 2005/123101, astringents, antiseptic agents, antistatics, binders, buffers, carrier materials, preferably those mentioned in WO 2005/123101, chelating agents, preferably those mentioned in WO 2005/123101, cell stimulants, cleansing agents, conditioning agents, depilatories, surface-active substances, deodorizing agents and antiperspirants, preferably those mentioned in WO 2005/123101, softeners, emulsifiers, preferably those mentioned in WO 2005/123101, enzymes, essential oils, preferably those mentioned in US 2008/0070825, insect repellants, preferably those mentioned in WO 2005/123101, fibers, film formers, fixatives, foam formers, foam stabilizers, anti-foam substances, foam boosters, fungicides, gelling agents and gel-forming agents, preferably those mentioned in WO 2005/123101, hair care products, hair shaping agents, hair smoothing products, moisture regulators (moisture-releasing substances, moisturizing substances and/or moisture-retaining substances), preferably those mentioned in WO 2005/123101, osmolytes, preferably those mentioned in WO 2005/123101, compatible solutes, preferably those mentioned in WO 01/76572 and WO 02/15686, bleaching agents, starching agents, stain-removing agents, optical brighteners, impregnating agents, dirt-repelling agents, friction-reducing agents, lubricants, moisturizing creams, ointments, opacifiers, plastizers, covering agents, polish, brighteners, polymers, preferably those mentioned in WO 2008/046676, powders, proteins and protein hydrolyzates, preferably those mentioned in WO 2005/123101 and WO 2008/046676, replumping agents, abrasive agents, skin-calming agents, skin cleansing agents, skin care agents, skin repair agents, preferably comprising cholesterol and/or fatty acids and/or ceramides and/or pseudoceramides, preferably those mentioned in WO 2006/053912, skin brightening agents, preferably those mentioned in WO 2007/110415, skin protection agents, skin softening agents, skin cooling agents, preferably those mentioned in WO 2005/123101, skin warming agents, preferably those mentioned in WO 2005/123101, stabilizers, UV absorbing agents and UV filters, preferably those mentioned in WO 2005/123101, benzylidene-beta-dicarbonyl compounds, preferably those mentioned in WO 2005/107692, alpha-benzoyl cinnamic acid nitriles, preferably those mentioned in WO 2006/015954, AhR receptor antagonists, preferably those mentioned in WO 2007/128723 and WO 2007/060256, detergents, fabric conditioners, suspending agents, skin tanning agents, preferably those mentioned in WO 2006/045760, thickeners, vitamins, preferably those mentioned in WO 2005/123101, oils, waxes and fats, preferably those mentioned in WO 2005/123101, phospholipids, preferably those mentioned in WO 2005/123101, fatty acids (saturated fatty acids, singly or polyunsaturated fatty acids, α-hydroxy acids, polyhydroxy fatty acids), preferably those mentioned in WO 2005/123101, liquefiers, dyes and color-protecting agents as well as pigments, preferably those mentioned in WO 2005/123101, anti-corrosives, aromas and flavorings as well as further additional fragrance substances, preferably the fragrance substances mentioned in S. Arctander, Perfume and Flavor Chemicals, private publishing house, Montclair, N.J., 1969 and Surburg, Panten, Common Fragrance and Flavor Materials, 5th Edition, Wiley-VCH, Weinheim 2006, in particular the further fragrance substances mentioned explicitly in US 2008/0070825 which are not already part of components (a) and (b) and optionally (c) and/or (d) of a fragrance substance mixture according to the invention or of a perfume oil according to the invention, alcohols and polyols, preferably those mentioned in WO 2005/123101, surfactants, preferably those mentioned in WO 2005/123101, animal extracts, yeast extracts, extracts of algae or micro-algae, electrolytes, liquefiers, organic solvents, preferably those mentioned in WO 2005/123101, or silicones and silicone derivatives, preferably those mentioned in WO 2008/046676.

Furthermore, fragrance substance mixtures according to the invention, in particular perfume oils, can be adsorbed on a carrier which ensures both a fine distribution of the fragrance substances comprised therein in the product and a controlled release during application. Carriers of this type can be porous inorganic materials, such as light sulfate, silica gels, zeolites, gypsum, clays, clay granulates, aerated concrete etc. or organic materials such as wood, cellulose-based substances, sugar, dextrins (for example maltodextrin) or plastics materials such as PVC, polyvinyl acetates or polyurethanes. The combination of composition according to the invention and carrier is one example item according to the invention.

Fragrance substance mixtures according to the invention, in particular perfume oils can also be present in micro-encapsulated form, spray-dried, as inclusion complexes or as extrusion products (i.e. products according to the invention) and can be added in this form, for example to a product to be perfumed.

If appropriate, the characteristics of the compositions modified in this manner can be further optimized by so-called “coating” with suitable materials in respect of a more targeted release of fragrance, for which purpose waxy plastics materials, for example polyvinyl alcohol, are preferably used. The resulting products are, again, products according to the invention.

The fragrance substance mixtures according to the invention, preferably the perfume oils according to the invention, can be micro-encapsulated, for example by the so-called coacervation process using encapsulating materials, consisting for example of polyurethane-like substances or soft gelatins. The spray-dried fragrance or flavoring substance compositions can be prepared for example by spray-drying an emulsion or dispersion comprising the fragrance substance mixture according to the invention, preferably a perfume oil, wherein the carriers used can be modified starches, proteins, dextrin and vegetable gums. Inclusion complexes can be prepared, for example by introducing dispersions of the fragrance substance mixture according to the invention, preferably a perfume oil according to the invention, and cyclodextrins or urea derivatives into a suitable solvent, for example water. Extrusion products can be obtained by melting a fragrance substance mixture according to the invention, preferably a perfume oil according to the invention, with a suitable waxy substance and by extrusion with subsequent solidification, optionally in a suitable solvent, for example isopropanol.

Fragrance substance mixtures according to the invention, preferably perfume oils according to the invention, can preferably be used as such, in concentrated form, in solutions or in a modified form described above for the production of perfumed products according to the invention.

Perfumed products which are preferred according to the invention comprise a fragrance substance mixture according to the invention, preferably a perfume oil according to the invention, in a sensorially effective amount.

The proportion of a fragrance substance mixture according to the invention, preferably of a perfume oil according to the invention, in perfumed products according to the invention is preferably from 0.01 to 10% by weight, preferably from 0.1 to 5% by weight and particularly preferably from 0.25 to 3% by weight, in each case based on the total mass of the perfumed product.

Preferred perfumed products according to the invention are laundry detergents and cleaning compositions, hygiene or care products, in particular in the field of body and hair care, cosmetics and household products.

Preferred perfumed products according to the invention are selected from the following list: perfume extracts, eaux de parfum, eaux de toilette, aftershaves, eaux de cologne, pre-shave products, splash colognes and perfumed freshening wipes, acidic, alkaline and neutral cleaning compositions, for example floor cleaners, window cleaners, dishwashing detergents, bath and sanitary cleaners, scouring milk, solid and liquid WC cleaners, pulverulent and foam carpet cleaners, textile fresheners, ironing aids, liquid detergents, pulverulent detergents, laundry pretreatment agents such as bleaches, soaking agents and stain removers, laundry conditioners, laundry soaps, laundry tablets, disinfectants, surface disinfectants, air fresheners in liquid or gel form or deposited on a solid carrier, aerosol sprays, waxes and polishes such as furniture polishes, floor waxes, shoe creams, bodycare compositions, for example solid and liquid soaps, shower gels, shampoos, shaving soaps, shaving foams, bath oils, cosmetic emulsions of the oil-in-water, water-in-oil and water-in-oil-in-water types, for example skin creams and lotions, face creams and lotions, sunscreen creams and lotions, aftersun creams and lotions, hand creams and lotions, foot creams and lotions, depilatory creams and lotions, aftershave creams and lotions, tanning creams and lotions, skin brightening creams and lotions, haircare products such as hair sprays, hair gels, hairsetting lotions, hair rinses, permanent and semi-permanent hair colorants, hair shaping compositions such as cold waves and hair smoothing compositions, hair tonics, hair creams and lotions, deodorants and antiperspirants, for example underarm sprays, roll-ons, deodorant sticks, deodorant creams, products for decorative cosmetics, for example eye shadows, nail varnishes, make-up, lipsticks, mascara as well as candles, lamp oils, joss-sticks, insecticides and repellents.

Perfumed products which are particularly preferred according to the invention are selected from the following list:
eaux de parfum, eaux de toilette, aftershaves, eaux de cologne, pre-shave products, splash colognes;
acidic, alkaline and neutral cleaning compositions, in particular in the household, preferably floor cleaners, window cleaners, dishwashing detergents, bath and sanitary cleaners, scouring milk, solid and liquid WC cleaners, pulverulent and foam carpet cleaners, liquid detergents, pulverulent detergents, laundry conditioners, surface disinfectants, in particular for hard surfaces (hard surface cleaners);
bodycare compositions, preferably solid and liquid soaps, shower gels, shampoos, shaving soaps, shaving foams;
cosmetic emulsions of the oil-in-water, water-in-oil and water-in-oil-in-water types, preferably skin creams and lotions, face creams and lotions, sun protection creams and lotions, aftersun creams and lotions, hand creams and lotions, foot creams and lotions, depilatory creams and lotions, aftershave creams and lotions, tanning creams and lotions, skin brightening creams and lotions;
haircare products, preferably hair sprays, hair gels, hairsetting lotions, hair rinses, permanent and semi-permanent hair colorants, hair tonics, hair creams and lotions;
deodorants and antiperspirants, preferably underarm sprays, roll-ons (preferably as an alcoholic or non-alcoholic solution, as a gel or (micro) emulsion), deodorant sticks, deodorant creams.

The following examples illustrate the invention; proportions and percentages relate to the weight, unless indicated otherwise.

### Abbreviations Used:

Dipropylene glycol (DPG), diethyl phthalate (DEP), triethyl citrate (TEC), isopropyl myristate (IPM); nat.=natural; macrocyclic musk fragrance substance (component (b) of the fragrance substance mixture according to the invention (MACRO)); polycyclic musk fragrance substance (component (b) of the fragrance substance mixture according to the invention (POLY)); alicyclic musk fragrance substance (component (b) of the fragrance substance mixture according to the invention (ALICYC));
HEDIONE® HC/30 comprises at least 30% by weight of the cis-isomer and just 70% by weight of the trans-isomer of methyl dihydrojasmonate.

For explanations of the product names of the fragrance substances, see, for example, S. Arctander, Perfume and Flavor Materials, Vol. I and II, Montclair, N.J., 1969, private publishing house or H. Surburg, J. Panten, “Common Fragrance and Flavor Materials”, 5^{th}. Ed., Wiley-VCH, Weinheim 2006.

### EXAMPLES

### Perfume Oil

### Example P1

| | | |
|---|---|---|
| AMBRETTOLIDE (MACRO) | 10.00 | 10.00 |
| AMBROXIDE 10% in IPM | 10.00 | 10.00 |
| BENZYL ACETATE | 20.00 | 20.00 |
| BENZYL SALICYLATE | 15.00 | 15.00 |
| BERGAMOT OIL, bergapten-free | 60.00 | 60.00 |
| CALONE ® 1951 10% in DPG | 15.00 | 15.00 |
| COUMARIN | 5.00 | 5.00 |
| CYCLOGALBANATE ® 10% in DPG | 10.00 | 10.00 |
| ALPHA-DAMASCONE 1% in DPG | 20.00 | 20.00 |
| DIHYDROMYRCENOL | 10.00 | 10.00 |
| ETHYL LINALOOL | 75.00 | 75.00 |
| ETHYL LINALYLACETATE | 50.00 | 50.00 |
| ETHYL MALTOL 1% in DEP | 10.00 | 10.00 |
| ETHYLENE BRASSYLATE | 80.00 | 80.00 |
| (MACRO) | | |
| FLOROSA | 40.00 | 40.00 |
| GERANYLACETATE | 10.00 | 10.00 |
| HEDIONE ® HC/30 | 35.00 | 35.00 |
| HEDIONE ® | 210.00 | 210.00 |
| HELIONAL ® | 15.00 | 15.00 |
| HELVETOLIDE ® (ALICYC) | 30.00 | 30.00 |
| HEXENYLSALICYLATE CIS-3 | 20.00 | 20.00 |
| ISO E SUPER ® | 40.00 | 40.00 |
| LEAFOVERT ® 10% in DEP | 10.00 | 10.00 |
| LILIAL ® | 80.00 | 80.00 |
| LYRAL ® | 20.00 | 20.00 |
| MANDARIN OIL | 10.00 | 10.00 |
| STYRALYL ACETATE | 5.00 | 5.00 |
| SYMROSE ® | 15.00 | 15.00 |
| VANILLIN 10% in DEP | 20.00 | 20.00 |
| DIPROPYLENE GLYCOL (DPG) | 50.00 | 20.00 |
| COMPOUND (A) | — | 30.00 |
| TOTAL: | 1,000.00 | 1,000.00 |

The addition of 3% of compound (A) to this floriental-citrus-musk composition produces a perceptible harmonization of the base notes, so that the entire composition seems more elegant and valuable.

### Perfume Oil

### Example P2

| | | |
|---|---|---|
| AMAROCITE ® | 10.00 | 10.00 |
| AMBROCENIDE ® 10% in DPG | 5.00 | 5.00 |
| AMBROXIDE | 15.00 | 15.00 |
| AURELIONE ® (⅞- | 70.00 | 70.00 |
| Cyclohexadecenone) (MACRO) | | |
| BERGAMOT OIL, bergapten-free | 90.00 | 90.00 |
| CALONE ® 1951 10% in DPG | 20.00 | 20.00 |
| CARAWAY OIL | 10.00 | 10.00 |
| CITRAL | 20.00 | 20.00 |
| COUMARIN | 10.00 | 1000 |
| ALPHA-DAMASCONE 1% in DPG | 15.00 | 15.00 |
| DIHYDROMYRCENOL | 70.00 | 70.00 |
| ESTRAGON OIL | 10.00 | 10.00 |
| ETHYL LINALOOL | 100.00 | 100.00 |
| ETHYL LINALYLACETATE | 90.00 | 90.00 |
| EUGENOL | 10.00 | 10.00 |
| EVERNYL ® | 5.00 | 5.00 |
| FRUCTATE ® | 5.00 | 5.00 |
| GERANIUM OIL | 5.00 | 5.00 |
| HEDIONE ® HC/30 | 100.00 | 100.00 |
| HELIONAL ® | 10.00 | 10.00 |
| INDOLE 10% in DPG | 5.00 | 5.00 |
| ISO E SUPER ® | 100.00 | 100.00 |
| KEPHALIS ® | 5.00 | 5.00 |
| LAVENDER OIL | 40.00 | 40.00 |
| CITRUS OIL | 80.00 | 80.00 |
| LILIAL ® | 30.00 | 30.00 |
| MANDARIN OIL | 20.00 | 20.00 |
| MUSCENONE (MACRO) | 5.00 | 5.00 |
| SANDRANOL ® | 10.00 | 10.00 |
| VANILLIN 10% in DPG | 5.00 | 5.00 |
| DIPROPYLENE GLYCOL | 30.00 | 10.00 |
| COMPOUND (A) | — | 20.00 |
| TOTAL: | 1,000.00 | 1,000.00 |

The addition of 2% of compound (A) produces, while interacting with Iso E Super®, a very valuable woody classic vetiver effect which makes the entire composition seem more valuable. In addition, there is a significant harmonization of the base notes, so that the entire composition seems more elegant.

### Perfume Oil

### Example P3

| | | |
|---|---|---|
| ALDEHYDE C10 (n-decanal) 10% in DPG | 20.00 | 20.00 |
| ALDEHYDE C11 (n-undecanal) 10% in DPG | 5.00 | 5.00 |
| ALDEHYDE C12 (n-dodecanal) 10% in DPG | 15.00 | 15.00 |
| AMBRETTOLIDE (MACRO) | 5.00 | 5.00 |
| AMBROCENIDE ® 1% in DPG | 20.00 | 20.00 |
| AURELIONE ® (MACRO) | 30.00 | 30.00 |
| BENZYL ACETATE | 30.00 | 30.00 |
| CITRONELLA OIL | 15.00 | 15.00 |
| ETHYL VANILLIN 1% in DPG | 20.00 | 20.00 |
| ETHYLENE BRASSYLATE (MACRO) | 70.00 | 70.00 |
| FRUCTATE ® 10% in DPG | 20.00 | 20.00 |
| GERANYL ACETATE | 10.00 | 10.00 |
| GLOBALIDE ® (MACRO) | 30.00 | 30.00 |
| HEDIONE ® | 30.00 | 30.00 |
| ALPHA-HEXYLCINNAMALDEHYDE | 90.00 | 90.00 |
| INDOLE 10% in DPG | 5.00 | 5.00 |
| ISO E SUPER ® | 120.00 | 120.00 |
| KEPHALIS ® | 5.00 | 5.00 |
| LINALOOL | 150.00 | 150.00 |
| LINALYL ACETATE | 60.00 | 60.00 |
| BETA-METHYLNAPHTYLKETONE | 5.00 | 5.00 |
| NEROLIDOL | 20.00 | 20.00 |
| NEROLIONE 10% in DPG | 20.00 | 20.00 |
| BRAZILIAN ORANGE OIL | 100.00 | 100.00 |
| PHENYLETHYL ACETATE | 5.00 | 5.00 |
| PHENYLETHYL ALCOHOL | 30.00 | 30.00 |
| TERPINEOL | 20.00 | 20.00 |
| DIPROPYLENE GLYCOL | 50.00 | 20.00 |
| COMPOUND (A) | — | 30.00 |
| TOTAL: | 1,000.00 | 1,000.00 |

The addition of 3% of compound (A) emphasizes the erogenous and animalic aspects of this composition and the accompanying harmonization of the base notes makes the entire composition seem more elegant, creamier and thus significantly more valuable.

### Perfume Oil

### Example P4

| | | |
|---|---|---|
| ALLYLAMYL GLYCOLATE | 10.00 | 10.00 |
| BERGAMOT OIL | 100.00 | 100.00 |
| CALONE ® 1951 10% in DPG | 30.00 | 30.00 |
| CASSIS BASE | 10.00 | 10.00 |
| CLOVE OIL | 5.00 | 5.00 |
| ALPHA-DAMASCONE 1% in DPG | 2000 | 20.00 |
| DIHYDROMYRCENOL | 60.00 | 60.00 |
| ETHYLENE BRASSYLATE (MACRO) | 20.00 | 20.00 |
| FARENAL ® 1% in DPG | 20.00 | 20.00 |
| FLORALOZONE ® | 10.00 | 10.00 |
| GLOBALIDE ® (MACRO) | 20.00 | 20.00 |
| HEDIONE ® HC/30 | 10.00 | 10.00 |
| HEDIONE ® | 200.00 | 200.00 |
| ALPHA-HEXYLCINNAMALDEHYDE | 100.00 | 100.00 |
| HEXYLSALICYLATE | 20.00 | 20.00 |
| ISO E SUPER ® | 70.00 | 70.00 |
| KEPHALIS | 5.00 | 5.00 |
| LAVANDER OIL GROSSO NAT. | 20.00 | 20.00 |
| LEAFOVERT ® 10% in DPG | 20.00 | 20.00 |
| LIGUSTRAL 10% in DPG | 10.00 | 10.00 |
| LILIAL ® | 45.00 | 45.00 |
| LINALOOL | 60.00 | 60.00 |
| LINALYL ACETATE | 50.00 | 50.00 |
| MANDARIN ALDEHYDE 10% in TEC | 5.00 | 5.00 |
| BRAZILIAN ORANGE OIL | 25.00 | 25.00 |
| POLYSANTOL ® | 5.00 | 5.00 |
| TERPINEOL | 20.00 | 20.00 |
| DIPROPYLENE GLYCOL | 30.00 | — |
| COMPOUND (A) | — | 30.00 |
| TOTAL: | 1,000.00 | 1,000.00 |

The addition of 3% of compound (A) emphasizes in a unique manner the transparency of the composition, when combined with the musk fragrance substances and methyl dihydrojasmonate and furthermore, the base notes are harmonized such that the entire composition seems more erogenous and more masculine and, overall, the effect of the entire composition is more valuable.

### Perfume Oil

### Example P5

| | | |
|---|---|---|
| Florazon | 15.00 | 15.00 |
| Cyclogalbanate ® | 15.00 | 15.00 |
| Bergamot oil | 135.00 | 135.00 |
| Mandarin oil | 50.00 | 50.00 |
| Claritone ® | 100.00 | 100.00 |
| Oxanthia 5% in DPG | 5.00 | 5.00 |
| Amarocit ® | 10.00 | 10.00 |
| Peppermint oil | 5.00 | 5.00 |
| Wormwood oil | 5.00 | 5.00 |
| Mace oil | 5.00 | 5.00 |
| Lilial ® | 50.00 | 50.00 |
| Calone ® 1951 10% in DPG | 25.00 | 25.00 |
| Geranium oil | 10.00 | 10.00 |
| Hedione ® | 240.00 | 240.00 |
| Hexenylsalicylate cis-3 | 15.00 | 15.00 |
| Parmanyl ® | 5.00 | 5.00 |
| Beta-ionone | 25.00 | 25.00 |
| Cedarwood oil | 30.00 | 30.00 |
| Iso E Super ® | 80.00 | 80.00 |
| Vetival ® | 40.00 | 40.00 |
| Vetikol acetate ® | 5.00 | 5.00 |
| Sandranol ® | 75.00 | 75.00 |
| Ysamber ® K | 10.00 | 10.00 |
| Globalide ® (MACRO) | 25.00 | 25.00 |
| Dipropylene glycol | 20.00 | 15.00 |
| Compound (A) | — | 5.00 |
| TOTAL: | 1,000.00 | 1,000.00 |

The addition of 0.5% of compound (A) emphasizes in a unique manner the earthy and woody aspects when combined with the musk fragrance substances and Iso E Super®. Furthermore, the fresh and spicy aspects are emphasized more strongly.

### Perfume Oil

### Example P6

| | | |
|---|---|---|
| Leafovert ® 10% in IPM | 10.00 | 10.00 |
| Bergamot oil | 50.00 | 50.00 |
| Orange oil | 40.00 | 40.00 |
| Oxanthia 10% in DPG | 5.00 | 5.00 |
| Black pepper oil 10% in | 20.00 | 20.00 |
| DPG | | |
| Camphor 10% in DPG | 5.00 | 5.00 |
| Gamma-decalactone 10% | 15.00 | 15.00 |
| in DPG | | |
| Gamma-undecalactone | 5.00 | 5.00 |
| 10% in DPG | | |
| Lilial ® | 60.00 | 60.00 |
| Helional | 25.00 | 25.00 |
| Linalool | 80.00 | 80.00 |
| Geranium oil 10% in DPG | 10.00 | 10.00 |
| Citronella oil | 20.00 | 20.00 |
| Geraniol | 15.00 | 15.00 |
| Isodamascone ® 10% in | 20.00 | 20.00 |
| DPG | | |
| Hedione ® | 120.00 | 120.00 |
| Alpha- | 40.00 | 40.00 |
| Hexylcinnamaldehyde | | |
| Vanillin | 15.00 | 15.00 |
| Coumarone ® | 5.00 | 5.00 |
| Iso E Super ® | 240.00 | 240.00 |
| Patchouli oil nat. | 25.00 | 25.00 |
| Sandalore | 10.00 | 10.00 |
| Labdanum Absolute 10% | 5.00 | 5.00 |
| in DPG | | |
| Ysambar ® K | 5.00 | 5.00 |
| Macrolide ® Supra | 25.00 | 25.00 |
| (MACRO) | | |
| Globalide ® (MACRO) | 45.00 | 45.00 |
| Cyclohexadecanone | 15.00 | 15.00 |
| (MACRO) | | |
| Ethylene brassylate | 45.00 | 45.00 |
| (MACRO) | | |
| Indole 10% in DPG | 5.00 | 5.00 |
| Dipropylene glycol | 20.00 | 10.00 |
| Compound (A) | — | 10.00 |
| TOTAL: | 1,000.00 | 1,000.00 |

The addition of 1.0% of compound (A) emphasizes the mossy aspects of the composition very nicely, in particular the interaction between the musk fragrance substances and Hedione®, as a result of which the effect of the composition is more harmonious and valuable.

### Perfume Oil

### Example P7

| | | |
|---|---|---|
| Styralyl acetate | 10.00 | 10.00 |
| Linalyl acetate | 40.00 | 40.00 |
| Mandaril 10% in DPG | 5.00 | 5.00 |
| Orange oil | 60.00 | 60.00 |
| Oxanthia 5% in DPG | 5.00 | 5.00 |
| Gamma-Decalactone | 5.00 | 5.00 |
| Ethyl-3-methyl-3-phenylglycidate | 5.00 | 5.00 |
| Frambinon ® | 5.00 | 5.00 |
| Raspberry base | 5.00 | 5.00 |
| Lilial ® | 80.00 | 80.00 |
| Calone ® 1951 10% in DPG | 20.00 | 20.00 |
| Majantol ® | 80.00 | 80.00 |
| Linalool | 60.00 | 60.00 |
| Phenylethyl alcohol | 20.00 | 20.00 |
| Citronellol | 20.00 | 20.00 |
| Geraniol | 5.00 | 5.00 |
| Benzyl acetate | 30.00 | 30.00 |
| Hedione ® | 150.00 | 150.00 |
| Alpha-Hexylcinnamaldehyde | 150.00 | 150.00 |
| Benzyl salicylate | 60.00 | 60.00 |
| Amyl salicylate (n- and iso-) | 40.00 | 40.00 |
| Beta-Ionone | 20.00 | 20.00 |
| Eugenol | 5.00 | 5.00 |
| Ethyl vanillin 10% in DPG | 10.00 | 10.00 |
| Cinnamyl alcohol | 5.00 | 5.00 |
| Iso E Super ® | 40.00 | 40.00 |
| Galaxolide ® 50% in DEP | 25.00 | 25.00 |
| (4,6,6,7,8,8-Hexamethyl-1,3,4,6,7,8- | | |
| hexahydrocyclopenta[g]benzopyrane) | | |
| (POLY) | | |
| Globalide ® (MACRO) | 20.00 | 20.00 |
| Dipropylene glycol | 20.00 | 15.00 |
| Compound (A) | — | 5.00 |
| TOTAL: | 1,000.00 | 1,000.00 |

The addition of 0.5% of compound (A) emphasizes the red-berry fruitiness of the composition and makes the entire composition seem more harmonious and more complex.

### Perfume Oil

### Example P8

| | | |
|---|---|---|
| Limonenal 10% in DPG | 6.00 | 6.00 |
| Profarnesal 10% in DPG | 4.00 | 4.00 |
| Hexenylbutyrate cis-3 | 2.00 | 2.00 |
| Mintonat ® | 3.00 | 3.00 |
| Linalyl acetate | 120.00 | 120.00 |
| Citrus oil | 20.00 | 20.00 |
| Citrylal | 2.00 | 2.00 |
| Gamma-Nonalactone | 15.00 | 15.00 |
| Frambinon ® | 2.00 | 2.00 |
| Raspberry base | 20.00 | 20.00 |
| Filbertone 1% in DPG | 1.00 | 1.00 |
| Acetylmethylcarbinol, nat. 10% in | 7.00 | 7.00 |
| DPG | | |
| Lilax Soft | 20.00 | 20.00 |
| Linalool | 30.00 | 30.00 |
| Terpinenol | 80.00 | 80.00 |
| Phenylethyl alcohol | 20.00 | 20.00 |
| Geraniol | 40.00 | 40.00 |
| Nerol | 70.00 | 70.00 |
| Citronellol | 30.00 | 30.00 |
| Benzyl acetate | 10.00 | 10.00 |
| Alpha-Amylcinnamaldehyde | 60.00 | 60.00 |
| Hedione ® | 130.00 | 130.00 |
| Beta-Ionone | 10.00 | 10.00 |
| Agrumex ® HC | 80.00 | 80.00 |
| Oryclon ® | 68.00 | 68.00 |
| Cedarwood oil | 2.00 | 2.00 |
| Vertofix ® | 120.00 | 120.00 |
| Macrolide ® (MACRO) | 20.00 | 20.00 |
| Triethyl citrate | 8.00 | 5.00 |
| Compound (A) | — | 3.00 |
| TOTAL: | 1,000.00 | 1,000.00 |

The addition of 0.3% of compound (A) emphasizes the red-berry fruitiness of the composition, in particular in the direction of blackberries and elderberries. Furthermore, the entire composition seems more harmonious and more complex.

The perfume oils P1, P2, P3, P4, P5, P6, P7 and P8 from the above perfume oil Examples P1 to P8 were worked separately in each case into the following formulations.

The olfactory effects which have been described above for the respective perfume oil were also observed in each case in the following formulations.

### Formulation Examples

### Example F1

### Washing Powder

| | | | % by | % by |
|---|---|---|---|---|
| Material | Chemical Name | Function | weight | weight |
| Sodium metasilicate | Sodium Metasilicate | | Ad 100 | Ad 100 |
| pentahydrate | Pentahydrate | | | |
| Sodium hydrogen-carbonate | Sodium Hydrogen | Alkali | 15.0 | 15.0 |
| | Carbonate | | | |
| Sodium percarbonate | Sodium Carbonate | Bleaching | 15.0 | 15.0 |
| | Peroxyhydrate | agent | | |
| Peractive AC Blue | TAED/ | Activator | 5.00 | 5.00 |
| | Na-Carboxymethylcellulose | | | |
| Genapol OA-080 | Oxoalcohol C14-15, 8EO | Non-ionic | 3.00 | 3.00 |
| | | surfactant | | |
| Texapon K12 powder | Sodium Lauryl Sulfate C12 | Anionic | 7.00 | 7.00 |
| | | surfactant | | |
| Tinopal CBS-X | | Brightener | 0.50 | 0.50 |
| Savinase 6.0 T, Type W | Protease | Enzyme | 0.40 | 0.40 |
| Termamyl 120 T | Alpha-Amylase | Enzyme | 0.30 | 030 |
| Sodium sulfate | Sodium Sulfate | Filler | 5.50 | 5.50 |
| Perfume oil P1, P2, | | Fragrance | 0.30 | 0.50 |
| P3, P4, P5, P6, P7 | | | | |
| and P8 | | | | |

### Example F2

### All-Purpose Cleaner

| | | | % by | % by |
|---|---|---|---|---|
| Material | Chemical Name | Function | weight | weight |
| Deionized | Water | Solvent | Ad 100 | Ad 100 |
| water | | | | |
| Mergal K9N | 5-Chloro-2-methyl- | Preservative | 0.1 | 0.1 |
| | 3-(2H)- | | | |
| | isothiazolone and | | | |
| | 2-methyl-3-(2H)- | | | |
| | isothiazolone | | | |
| Trisodium | Tri Sodium Citrate | Complex | 3.0 | 3.0 |
| citrate | Dihydrate | former | | |
| dihydrate | | | | |
| Zetesol NL-2 | Fatty alcohol C12-14- | Anionic | 30.0 | 30.0 |
| | sulfate, Sodium | surfactant | | |
| Imbentin | Fatty alcohol C12-C15, | Non-ionic | 5.0 | 5.0 |
| C/125/055 | 8EO | surfactant | | |
| Ethanol | Ethanol | Solvent | 2.0 | 2.0 |
| Perfume oil | | Fragrance | 0.3 | 0.5 |
| P1, P2, P3, | | | | |
| P4, P5, P6, | | | | |
| P7 and P8 | | | | |

### Example F3

### Shampoo

| | | % by | % by |
|---|---|---|---|
| Material | INCI Name | weight | weight |
| Deionized | Water | Ad 100 | Ad 100 |
| water | | | |
| Plantacare PS | Sodium Laureth Sulfate, Lauryl | 20.0 | 20.0 |
| 10 | Glucoside | | |
| Euperlan PK | Glycol Distearate, Sodium | 6.0 | 6.0 |
| 771 | Lauryl Sulfate, Cocamide MEA, | | |
| | Laureth-10 | | |
| Dragocid Liquid | Phenoxyethanol, | 0.5 | 0.5 |
| | Methylparaben, Ethylparaben, | | |
| | Butylparaben, Propylparaben, | | |
| | Isobutylparaben | | |
| Sodium | Sodium Chloride | 1.4 | 1.4 |
| chloride | | | |
| Citric acid | Citric acid | 0.1 | 0.1 |
| monohydrate | | | |
| crystalline | | | |
| Perfume oil P1, | Fragrance | 0.5 | 0.8 |
| P2, P3, P4, P5, | | | |
| P6, P7 and P8 | | | |

### Example F4

### Shower Gel

| | | % by | % by |
|---|---|---|---|
| Material | INCI Name | weight | weight |
| Deionized | Water | Ad 100 | Ad 100 |
| water | | | |
| Plantacare PS | Sodium Laureth Sulfate, Lauryl | 20.0 | 20.0 |
| 10 | Glucoside | | |
| Dragocid Liquid | Phenoxyethanol, | 0.5 | 0.5 |
| | Methylparaben, Ethylparaben, | | |
| | Butylparaben, Propylparaben, | | |
| | Isobutylparaben | | |
| Sodium | Sodium Chloride | 1.4 | 1.4 |
| chloride | | | |
| Citric acid | Citric Acid | 1.3 | 1.3 |
| monohydrate | | | |
| crystalline | | | |
| Perfume oil P1, | Fragrance | 0.5 | 0.7 |
| P2, P3, P4, P5, | | | |
| P6, P7 and P8 | | | |

### Example F5

### Fabric Conditioner

| | | | % by | By |
|---|---|---|---|---|
| Material | Chemical Name | Function | weight | weight |
| Deionized | Water | Solvent | Ad 100 | Ad 100 |
| water | | | | |
| Rewoquat | Dialkylesterammonium- | Cationic | 16.6 | 16.6 |
| WE 18 | ethosulfate | surfactant | | |
| Mergal K9N | 5-Chloro-2-methyl-3- | Preservative | 0.10 | 0.10 |
| | (2H)-isothiazolone and | | | |
| | 2-methyl-3-(2H)- | | | |
| | isothiazolone | | | |
| Dow | Polydimethyl-siloxane | Defoaming | 0.30 | 0.30 |
| Corning | | agent | | |
| 1520 | | | | |
| Antifoam | | | | |
| Magnesium | Magnesium Chloride | Consistency | 10.00 | 10.00 |
| chloride 1% | solution | provider | | |
| solution | | | | |
| Perfume oil | | Fragrance | 0.55 | 0.75 |
| P1, P2, P3, | | | | |
| P4, P5, P6, | | | | |
| P7 and P8 | | | | |

### Example F6

### Eau de Cologne/Eau de Toilette

| | % by | % by |
|---|---|---|
| Ingredients | weight | weight |
| Perfume oil P1, P2, P3, P4, | 5 | 10 |
| P5, P6, P7 and P8 | | |
| Ethanol | Ad 100 | Ad 100 |
| Water | 18 | 10 |

### Example F7

### Aerosol-Pump Spray

| | % by | % by |
|---|---|---|
| Ingredients | weight | weight |
| Perfume oil P1, P2, P3, P4, P5, | 1.0 | 1.5 |
| P6, P7 and P8 | | |
| Ethanol | Ad 100 | Ad 100 |
| Water | 5.0 | 8.0 |
| Alpha-Tocopherol | 0.20 | 0.20 |
| Hydroxypropyl cellulose | 0.20 | — |
| Rosemary extract, soluble in ethanol | 0.22 | — |
| Cetyl alcohol | 1.00 | 0.50 |

### Example F8

### Shampoo

| | % by | % by | % by |
|---|---|---|---|
| Ingredients | weight | weight | weight |
| Sodium lauryl ether sulfate (e.g. Texapon | 12 | 12 | 12 |
| NSO, produced by Cognis Deutschland | | | |
| GmbH) | | | |
| Cocamidopropyl betaine (e.g. Dehyton K, | 2 | 2 | 2 |
| produced by Cognis Deutschland GmbH) | | | |
| Sodium chloride | 1.4 | 1.4 | 1.4 |
| Citric acid | 1.3 | 1.3 | 1.3 |
| Phenoxyethanol, methyl-, ethyl-, butyl- | 0.5 | 0.5 | 0.5 |
| and propylparaben | | | |
| Perfume oil P1, P2, P3, P4, P5, P6, P7 | 0.3 | 0.5 | 0.7 |
| and P8 | | | |
| Water | Ad 100 | Ad 100 | Ad 100 |

### Example F9

### Washing Powder

| | % by | % by | % by |
|---|---|---|---|
| Ingredients | weight | weight | weight |
| Linear Na-Alkylbenzene sulfonate | 8.8 | 8.8 | 8.8 |
| Ethoxylated fatty alcohol C12-18 (7 EO) | 4.7 | 4.7 | 4.7 |
| Na-Soap | 3.2 | 3.2 | 3.2 |
| Defoamer DOW CORNING(R) | 3.9 | 3.9 | 3.9 |
| 2-4248S POWDERED ANTIFOAM, | | | |
| Silicone oil on zeolite as carrier | | | |
| Zeolite 4A | Ad 100 | Ad 100 | Ad 100 |
| Na-carbonate | 11.6 | 11.6 | 11.6 |
| Na-salt of a copolymer of acrylic acid and | 2.4 | 2.4 | 2.4 |
| maleic acid (Sokalan CP5) | | | |
| Na-Silicate | 3.0 | 3.0 | 3.0 |
| Carboxymethylcellulose | 1.2 | 1.2 | 1.2 |
| Dequest | 2.8 | 2.8 | 2.8 |
| 2066([[(Phosphonomethyl)imino]bis- | | | |
| [(ethylene-nitrilo)bis (methylene)]]tetrakis- | | | |
| phosphonic acid, sodium salt) | | | |
| Optical brightener | 0.2 | 0.2 | 0.2 |
| Na-Sulfate | 6.5 | 6.5 | 6.5 |
| Protease | 0.4 | 0.4 | 0.4 |
| Sodium perborate tetrahydrate | 21.7 | 21.7 | 21.7 |
| Perfume oil P1, P2, P3, P4, P5, P6, | 0.25 | 0.35 | 0.5 |
| P7 and P8 | | | |
| EDTA | 1.0 | 1.0 | 1.0 |

### Example F10

### Liquid Detergent

| | % by |
|---|---|
| Ingredients | weight |
| Deionized water | 39.9 |
| Optical brightener | 0.10 |
| Coconut fatty acids (C12-C18) | 7.5 |
| Potassium hydroxide 50% solution | 4.3 |
| Propane-1,2-diol | 5.00 |
| Fatty alcohols C12-C15, 8 EO | 12.0 |
| Na-salt of secondary alkyl sulfonates (C13-C17) | 17.0 |
| Triethanolamine | 2.0 |
| Trisodium citrate dihydrate | 5.0 |
| Dequest | 3.0 |
| 2066([[(Phosphonomethyl)imino]bis[(ethylenenitrilo)bis | |
| (methylene)]]tetrakis-phosphonic acid, sodium salt) | |
| Ethanol | 3.0 |
| Enzymes | 0.7 |
| Perfume oils P1, P2, P3, P4, P5, P6, P7 and P8 | 0.5 |

### Example F11

### Liquid Detergent Concentrate

| | % by |
|---|---|
| Ingredients | weight |
| Deionized water | 13.4 |
| Coconut fatty acids (C12-C18) | 10.0 |
| Fatty alcohols C12-C15, 8 EO | 26.0 |
| Na-salt of secondary alkyl sulfonates (C13-C17) | 26.5 |
| Triethanol amine | 8.5 |
| Na-salt of fatty alcohol sulfates C12-C14 | 3.0 |
| Ethanol | 5.5 |
| Urea | 4.5 |
| Enzymes | 0.9 |
| Citric acid | 1.0 |
| Perfume oil P1, P2, P3, P4, P5, P6, P7 and P8 | 0.7 |

1. A fragrance substance mixture comprising
(a) compound (A) 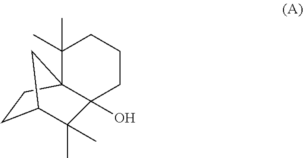
and
(b) at least one musk fragrance substance,
wherein
a mass ratio of a total quantity of musk fragrance substance or substances of component (b) to compound (A) (component (a)) is greater than or equal to 1:1.

2. The fragrance substance mixture as claimed in claim 1, wherein the musk fragrance substance or substances of component (b) are selected from the group consisting of macrocyclic musk fragrance substances, nitro-musk fragrance substances, polycyclic musk fragrance substances, alicyclic musk fragrance substances, and mixtures thereof.

3. The fragrance substance mixture according to claim 1 wherein component (b) comprises two, three or more different musk fragrance substances as component (b).

4. The fragrance substance mixture as claimed in claim 1, wherein the mass ratio of the total quantity of musk fragrance substances of component (b) to compound (A) (component (a)) is from 25:1 to 1:1.

5. The fragrance substance mixture as claimed in claim 1, further comprising
(c) one, two, three, four, five or more fragrance substances with a molecular weight of more than or equal to 190 g/mol which are not compound (A) (component (a)), and are not part of component (b),
and/or
(d) one, two, three or more fruity fragrance substances, wherein the fruity fragrance substance or substances is/are not part of component (a) and (b).

6. The fragrance substance mixture as claimed in claim 5, wherein component (c) comprises
(i) 2,3,8,8-tetramethyl-1,2,3,4,5,6,7,8-octahydro-2-naphthalenylmethylketone and/or 1-[1,2,3,4,6,7,8,8a-octahydro-1,2,8,8-tetramethylnaphthalen-2-yl]ethanone
and/or
(ii) methyl dihydrojasmonate.

7. The fragrance substance mixture as claimed in claim 6, wherein a mass ratio of a total quantity of component
(c) (i) 2,3,8,8-tetramethyl-1,2,3,4,5,6,7,8-octahydro-2-naphthalenylmethylketone and/or 1-[1,2,3,4,6,7,8,8a-octahydro-1,2,8,8-tetramethylnaphthalen-2-yl]ethanone to compound (A) (component (a)) is greater than or equal to 1:1.

8. The fragrance substance mixture according to claim 6, wherein a mass ratio of a total quantity of component
(c) (ii) methyl dihydrojasmonate to compound (A) (component (a)) is greater than or equal to 1:1.

9. The fragrance substance mixture as claimed in claim 1, wherein component
(b) comprises one or more macrocyclic musk fragrance substances.

10. The fragrance substance mixture as claimed in claim 1, wherein component
(b) comprises 15-pentadec-(11/12)-enolide, ethylene brassylate and/or oxacyclohexadecan-2-one.

11. A process for producing a fragrance substance mixture as claimed in claim 1, comprising:
mixing components (a) and (b), and optionally component
(c) one, two, three, four, five or more fragrance substances with a molecular weight of more than or equal to 190 g/mol, which are not compound (A) (component (a)), and are not part of component (b),
and/or component
(d) one, two, three or more fruity fragrance substances, wherein the fruity fragrance substance or substances is/are not part of component (a) and (b).

12. A process for emphasizing the erogenous, masculine and/or animalic odor impression of one or more musk compounds, comprising:
mixing one or more musk compounds with a quantity of compound (A), as claimed in claim 1, in an amount sufficient to emphasize the erogenous, masculine and/or animalic odor impression of one, more or all the musk compounds.

13. A process for emphasizing an odor with a woody, vetiver, creamy, earthy, fresh, spicy, mossy, (red) fruity, transparent and/or Fleur d'Orange note, comprising:
mixing
(i) one or more fragrance substances, having one, more or all the woody, vetiver, creamy, earthy, fresh, spicy, mossy, (red) fruity, transparent and/or Fleur d'Orange notes,
and
(ii) one or more musk fragrance substances, or
(ii) a fragrance substance mixture as claimed in claim 1, in an amount sufficient to emphasize the odor impression of the fragrance substance or substances which produce one, more or all the woody, vetiver, creamy, earthy, fresh, spicy, mossy, (red) fruity, transparent and/or Fleur d'Orange notes,
with a quantity of compound (A) (component (a)) which is in an amount sufficient to emphasize the odor impression of the fragrance substance or substances which produce one, more or all the woody, vetiver, creamy, earthy, fresh, spicy, mossy, (red) fruity, transparent and/or Fleur d'Orange notes.

14. A perfumed product comprising a fragrance substance mixture as claimed in claim 1.

15. The perfumed product as claimed in claim 14, selected from the group consisting of laundry detergents, cleaning compositions, hygiene or care products, cosmetics, and household products.

16. The fragrance substance mixture as claimed in claim 1, wherein the mass ratio of the total quantity of musk fragrance substances of component (b) to compound (A) (component (a)) is from 1:1 to 50:1.

17. The fragrance substance mixture as claimed in claim 1, wherein the mass ratio of the total quantity of musk fragrance substances of component (b) to compound (A) (component (a)) is from 4:3 to 15:1.

18. The fragrance substance mixture as claimed in claim 1, wherein the mass ratio of the total quantity of musk fragrance substances of component (b) to compound (A) (component (a)) is 2:1 to 10:1.

19. A perfume oil comprising the fragrance substance mixture as claimed in claim 1.

20. The fragrance substance mixture as claimed in claim 9, wherein the one or more macrocyclic musk fragrance substances are selected from the group consisting of macrocyclic C₁₄-C₁₈ ketones and macrocyclic C₁₄-C₁₈ lactones.
